(19) United States
(12) Patent Application Publication
(10) Pub. No.: US 2015/0104399 A1
(43) Pub. Date: Apr. 16, 2015

(54) PREVENTION OF FIBROBLAST COLLAPSE

(71) Applicant: Biocogent, LLC, Stony Brook, NY (US)

(72) Inventors: Joseph D. Ceccoli, Farmingville, NY (US); Brian Costello, Port Jefferson Station, NY (US)

(73) Assignee: Biocogent, LLC, Stony Brook, NY (US)

(21) Appl. No.: 14/401,080

(22) PCT Filed: May 14, 2013
(86) PCT No.: PCT/US2013/040972

§ 371 Date: Nov. 13, 2014

Related U.S. Application Data
(60) Provisional application No. 61/646,711, filed on May 14, 2012.

(51) Int. Cl. A61K 8/97

(52) U.S. Cl. CPC A61K 8/97; A61Q 19/08
USPC 424/59; 424/769

(57) ABSTRACT

The present technology is directed to extracts of plants of genus Osmanthus, skin care compositions comprising extracts of plants of the genus Osmanthus; as well as to methods of formulating skin care compositions, and methods of reducing or preventing fibroblast collapse, reducing or preventing glycation, maintaining or increasing skin elasticity and firmness and reducing the appearance of aging comprising applying the skin care compositions of the present technology to the skin of a patient.

### CROSS-REFERENCE TO RELATED APPLICATIONS

The present application claims the benefit of U.S. Provisional Patent Application No. 61/646,711 filed May 14, 2012, the contents of which are incorporated by reference in its entirety.

### TECHNICAL FIELD

This technology relates generally to personal care compositions, specifically skin care compositions that are useful in the prevention of fibroblast collapse and resultant aging and appearance of aging, as well as skin care compositions comprising extracts of plants of the genus Osmanthus and methods of using such extracts.

### BACKGROUND

Collagen and elastin are important protein ingredients in healthy skin, and are derived from procollagen and tropoelastin, respectively, which are produced and processed by fibroblasts. Sufficient quantities of properly organized collagen and elastin are necessary in the skin to maintain youthful physical properties such as tensile strength and elasticity and to support a youthful appearance of the skin without visible wrinkling and laxity. Fibroblast collapse is a process that is known to occur in conjunction with aging of the skin. The term fibroblast collapse has been used to refer to the literal physical collapse or shrinking of individual fibroblast cells as a result of loss of proper attachment to the extracellular matrix which is their normal substrate. This occurs due to abnormal structural changes of the extracellular matrix that are initiated by glycation and consequent formation of advanced glycation endproducts (referred to herein as “AGEs”). Fibroblast collapse also can refer to the collapse of the fibroblast population in the skin, that is to say to a decrease in the number of fibroblasts in the skin due to the toxic effects of advanced glycation endproducts (AGEs).

Glycation refers to the bonding of a protein in the skin to a sugar molecule, or to certain reactive carbonyl compounds derived from metabolic reactions, or from oxidation of lipid. Glycation is an instrumental process in leading to fibroblast collapse, and consequently the aging of skin and other tissues in the body. Glycation can be hastened by high amounts of sugar in the body (generally as a result of diabetes or a high-sugar diet). Glycation can lead to impairment of molecular function, and eventually to decreased elasticity and tensile strength, decreased firmness, and to increased aging of the skin. Once glycated, body tissues produce advanced glycation end products (AGEs), which lead to oxidative damage, abnormal crosslinking of proteins, cytotoxicity, tissue breakdown and inflammation.

Osmanthus are a genus of flowering plants that are native to Asia and North America, and whose flowers have been found to be useful in the manufacture of teas and perfumes. However, it has herein been discovered for the first time that certain skin care compositions comprising extracts of plants of the genus Osmanthus, in particular Osmanthus fragrans, are particularly useful in preventing fibroblast collapse and protecting the body against the toxic effects of glycation when applied to the skin.

In particular, Osmanthus extracts comprise, among other ingredients, flavonoids including luteolin, carotenoids such as beta-carotene, and many small fragrance molecules such as the terpene alcohol linalool, many of which are thought to have antioxidant and anti-inflammatory properties.

As of now, it has not previously been contemplated to use such extracts in skin care compositions. Nor has it been contemplated to use such compositions topically on the skin to prevent the effects of glycation on cellular breakdown and aging.

The present technology provides the advantage of providing such compositions as well as methods of using such compositions to prevent and decrease fibroblast collapse, and therefore prevent premature aging, the appearance of premature aging and tissue damage.

### BRIEF SUMMARY

In certain embodiments, the present technology is directed to a skin care composition comprising an extract of a plant of genus Osmanthus.

In other embodiments, the present technology is directed to a skin care composition comprising: (a) about 0.001 to about 50% of an extract of a plant of genus Osmanthus; (b) about 0.1 to about 20% of an emulsifier; (c) about 0.1 to about 60% of an emollient; (d) about 0.01 to about 10% of a thickener; and (e) about 0.1 to about 99% water.

In other embodiments, the present technology is directed to a method of formulating a skin care composition, the method comprising the steps of: (a) preparing an extract of a plant of genus Osmanthus by preparing a combination of about 2 to about 10 parts by weight of any portion of the plant with about 90 to about 98 parts by weight of solvent; and (b) combining the extract with a skin care ingredient to produce the skin care composition.

The present technology is directed, in other embodiments, to methods of reducing or preventing fibroblast collapse, reducing or preventing glycation, maintaining or increasing skin elasticity and tensile strength, methods of increasing firmness and methods of reducing the appearance of aging, comprising applying the skin care compositions of the present technology to the skin of a patient.

### BRIEF DESCRIPTION OF THE DRAWINGS

FIG. 1 shows a comparison of the amounts of AGE-modified protein among different compositions, including two compositions comprising an extract of a plant of genus Osmanthus.

FIG. 2 shows a comparison of the amounts of AGE-modified protein among different compositions, including three additional compositions comprising an extract of a plant of genus Osmanthus.

FIG. 3 shows a comparison of Percentage of Cell Viability among different compositions.

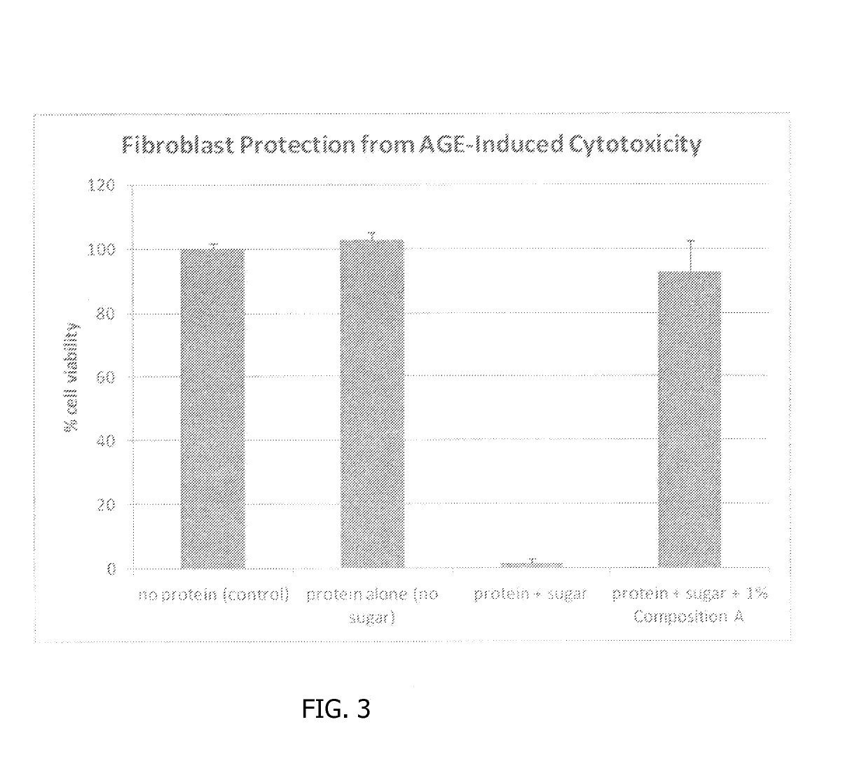

FIG. 4 shows the relative strength and number of healthy fibroblasts exposed to a protein sample that has not been contacted with a reducing sugar.

FIG. 5 shows the relative strength and number of healthy fibroblasts exposed to a protein sample that has been contacted with a reducing sugar.

FIG. 6 shows the relative strength and number of healthy fibroblasts exposed to a protein sample that has been contacted with a reducing sugar in the presence of pyridoxamine.

FIG. 7 shows the relative strength and number of healthy fibroblasts exposed to a protein sample that has been contacted with a reducing sugar in the presence of 1% Composition A, which includes an extract of Osmanthus according to the present technology.

### DETAILED DESCRIPTION

As described herein, in certain embodiments, the present technology is directed to skin care compositions comprising extracts of the genus Osmanthus. In certain embodiments, such compositions include, but are not limited to, known species such as the following: Osmanthus americanus, Osmanthus amatus, Osmanthus asiaticus (sweet olive), Osmanthus auratiacus, Osmanthus decorus, Osmanthus delavayi, Osmanthus fragrans, Osmanthus heterophyllus, Osmanthus semulatus, Osmanthus suavis, Osmanthus yunnanensis, as well as garden hybrids such as, e.g., Osmanthus×burkwoodii (O. delavayi×O. decorus) and Osmanthus×fortunei (O. fragrans×O. heterophyllus).

It has herein been discovered that skin care compositions comprising an extract of Osmanthus are beneficial when applied, in various embodiments, topically to the skin or to skin fibroblasts. In particular, extracts of Osmanthus fragrans are optimal in certain embodiments. As used herein, “extract” refers to an extract of any part of the plant (including without limitation, flowers, stems, roots, seeds, fruit or leaves) that results from a chemical or physical separation process using any solvent, including water. Throughout this disclosure, reference will be made to “Composition A” which is an example of a composition that has been prepared in accordance with the methods discussed herein, and that may include varying concentrations of the actual Osmanthus extract along with other materials such as water or other solvents. As used herein, “plant” refers to any of these or other portions of the plant.

A current study of the extracts of Osmanthus show that such extracts have been found to include, among other ingredients, one or more of the following components:
Octane
5-octen-1-ol
trans-linalool oxide (furan and pyran)
cis-linalool oxide (furan and pyran)
linalool
nonanal
2-methylhexanoic acid
Lilac alcohol
Decanal
Beta-ionol
Nerol
10-pentadecen-1-ol
Alpha-ionone
Dihydro-beta-ionone
Gamma-decalactone
Beta-ionone
5,6,7,7-tetrahydro-4,4,7-trimethyl-2(4H)-benzofuranone
Hexadecane
4-hydroxy-beta-ionone
3-oxo-beta-ionone
3-(3-hydroxybutyl)-2,2,4-trimethyl-2-cyclohexen-1-one
9-octadecanoic acid
2,6,10-trimethyltetradecane
6,10,14-trimethyl-2-pentadecanone
Dibutyl phthalate
Hexadecanoic acid
Hexadecanoic acid ethyl ester
Octadecanal
9,12,15-octadecatrienoic acid
9,12,15-octadecatrienoic acid, methyl ester
Docosane
1-docosanol
All trans-beta-carotene
All trans-alpha-carotene
Neo-beta-carotene B
Cis-jasmone
Gamma-decalectone
Various delta lactones
Linoleic acid
Linolenic acid
Palmitic acid
Oleic acid
Ethyl linolenate
(+)-decan-4-olide
Ethyl palmitate
Ethyl linoleate
Dihydro-beta-ionol
Stearic acid
Trans-geranic acid
Eicosanol
Ethyl oleate
Geraniol
p-methoxyphenylethanol
tetradecanoic acid
4-oxo-dihydro-beta-ionol
Retroionenes (4 isomers)
Ethyl stearate
(+)-Theaspirane B (trans)
p-ethylphenol
(+)-Theaspirane A (cis)
4-hydroxy-beta-ionol
4-oxo-beta-ionol
4-oxo-dihydro-beta-ionone
6-pentyl-alpha-pyrone
Ethyl tetradecanoate
(−)-7-oxodihydrotheaspirane B1
(+)-7-oxodihydrotheaspirane A1
(−)-7-oxodihydrotheaspirane A2
(+)-7-oxodihydrotheaspirane B2
(+)-nerolidol
2-phenylethanol
4-oxo-beta-ionone
1-nonanol
7(Z)-decene-4-olide
7(Z)-decene-5-olide
Citronellol
Dodecan-4-olide
Eugenol
2(3)-dehydrotheaspirane
1-(2,6,6-trimethyl-1,3-cyclohexadiene-1-yl)-3-butanone
Benzyl alcohol
Damascenone
Ethyl nonanoate
Ethyl octanoate
p-menth-1-en-9-al
photoisomer of beta ionone
1-(2,6,6-trimethyl-1,3-cyclohexadiene-1-yl)-3-butanol
Beta-ionone epoxide
Decan-5-olide
Phenylacetylnitrile
(E)-retroionol
(E)-retroionone
1-(2,3,6-trimethylphenyl)but-1-en-3-one
2(Z),7(Z)-decadien-5-olide
5(Z), 8(Z), 11(Z)-tetradecatrien-4-olide
2,5-epoxy-megastigma-6(E),8(E)-diene
Alpha-ionol
Alpha-terpineol
Beta-ionyl ethyl ether
Coumarin
Hexadecane-4-olide
Hotrienol
Megastigma-5,7(E),9-triene-4-one
Nerol oxide
Nonan-4-olide
Octan-4-olide
(Z)-retroionol
(Z)-retroionone
2-decene-5-olide
2-methyl-2-vinyl-5-(2′-6-methylhepta-2,6-dienyl)furan
3-oxo-alpha-ionone
4-oxo-isophorone
5(Z),8(Z)-tetradecadien-4-olide
5(Z)-tetradecan-4-olide
6,7-epoxy-theaspirane
6-heptyl-alpha-pyrone
6-hydroxy-dihydrotheaspirane
6-propyl-alpha-pyrone
Cis-7,10-epoxy-2,6,10-trimethyl-2,5(E),11-dodecatriene
trans-7,10-epoxy-2,6,10-trimethyl-2,5(E),11-dodecatriene
Geranyl acetate
Megastigma-4,6,8-triene-3-ones (4 isomers)
Megastigma-4,7(E),9-triene-3-one
Megastigma-5,8(E)diene-4-one
2,5-epoxy-megastigma-6(Z),8(E)-diene
2,3-epoxy-4-oxo-isophorone
2,6,6-trimethylcyclohex-1-en-carboxylic acid
2-Hydroxy-2,6,6-trimethylcyclohexanone
3-Oxo-retroionol
9-Oxo-dihydroeudalan
9-Oxo-eudalan
Beta-cyclocitral
Beta-damascone
Dihydroactinodiolide
Isophorone (3,5,5-trimethylcyclohex-2-en-1-one)
Safranal
Theaspirone
1-(2,3,6-Trimethylphenyl)but-1-en-3-ethoxy ether
2,3,6-Trimethylphenylbutan-3-ol
2,5-Epoxy-6-megastigmen-9-ol
2,7-Epoxy-megastigma-4,8(E)-diene
2-Methyl-2-vinyl-5-isopropenyfuran (2-isomers)
4,7-Epoxy-megastigma-5 (11),8(E)-diene
9-Hydroxytheaspirane
Dehydro-ar-ionone
Hexan-4-olide

As used herein, “skin care compositions” refer to compositions that are applied topically to the skin. As used herein, “skin” refers to the soft tissues covering the outer surface of a human or mammal, and includes the hair, scalp and nails. In certain embodiments of the present technology, the skin care compositions discussed herein can refer to any such compositions other than compositions that are solely perfumes or fragrances. As used herein, skin care compositions include, but are not limited to, lotions, creams, pastes, suspensions, gels, liquids, aerosols, powders, foams, ointments, serums, sprays, sachets or mixtures of any of the foregoing.

As used herein, all components of compositions expressed in percentages refer to percentages by weight, unless otherwise noted.

In certain embodiments, the present technology contemplates an extract of a plant of genus Osmanthus, as well as skin care compositions comprising such extract. Such extract may be obtained by contacting the plant with water or another solvent. Examples of useful solvents in both obtaining the extract and adding to the skin care compositions discussed herein include organic solvents such as, for example, glycerin, propanediol, propylene glycol, ethylene glycol, pentane, cyclopentane, hexane, hexanediol, cyclohexane, benzene, toluene, dioxane, diethyl ether, chloroform, dichloromethane (DCM), tetrahydrofuran (THF), ethyl acetate, acetone, dimethylformamide (DMF), acetonitrile, dimethyl sulfoxide (DMSO), propylene carbonate, formic acid, butanol, propanol, isopropanol, ethanol, methanol, acetic acid, nitromethane or the like.

In an exemplary method, an extract of Osmanthus may be prepared as follows: in various embodiments, any part of the Osmanthus plant may be obtained and combined with water. For example, by weight about 1 to about 75 parts, about 1 to about 50 parts, about 1 to about 25 parts, about 1 to about 10 parts, about 3, 4, 5 or about 10 parts of the Osmanthus plant may be combined with a corresponding amount of water or any other solvent, such that the total parts equals 100. This combination is then heated up in certain embodiments (although heating is not required) and optionally filtered to remove particulates, yielding a material (referred to herein as “Composition A” for ease of reference). Depending on the relative amounts of Osmanthus and solvent combined, Composition A may contain, in various embodiments, about 0.01 to about 95%, about 0.01 to about 90%, about 0.01 to about 75%, about 0.01 to about 50%, about 0.01 to about 25% or about 0.01 to about 15% of the desirable Osmanthus extract.

In certain embodiments, an Osmanthus extract discussed herein may be in liquid or substantially liquid form, as described herein; or it may be in solid or substantially solid form, for example, a powder or pulverized material, granule, tablet, a cream, grounds, an emulsion, a suspension or the like. For example, the extract may be dried and then milled to powder, or can be used to produce a paste that can also optionally be dried. The content of the active ingredient(s) in the extract can be measured using, for example, HPLC, IN or other spectrometry methods.

An Osmanthus extract discussed herein, regardless of form, may then, in certain embodiments, be combined with an additional ingredient (referred to herein as a “skin care ingredient”) chosen from one or more of the following: emollients, moisturizers, surfactants, polymers, vitamins, botanical extracts, solvents (including water and organic solvents), lipids (including fats and oils), sunscreen agents, exfoliating agents, colorants, maskants, or perfumes or fragrances, healing agents, skin anti-aging agents, skin moisturizing agents, anti-wrinkle agents, anti-atrophy agents, skin smoothing agents, antibacterial agents, antifungal agents, pesticides anti parasitic agents, antimicrobial agents, anti-inflammatory agents, anti-pruriginous agents, external anesthetic agents, antiviral agents, keratolytic agents, free radicals scavengers, antiseborrheic agents, antidandruff agents, the agents modulating the differentiation, proliferation or pigmentation of the skin and agents accelerating penetration, desquamating agents, depigmenting or propigmenting agents, antiglycation agents, tightening agents, agents stimulating the synthesis of dermal or epidermal macromolecules and/or preventing their degradation; agents stimulating the proliferation of fibroblasts and/or keratinocytes or stimulating the differentiation of keratinocytes; muscle relaxants; antipollution and/or anti-free radical agents; slimming agents, anticellulite agents, agents acting on the microcirculation; agents acting on the energy metabolism of the cells; cleaning agents, hair conditioning agents, hair styling agents, hair growth promoters, sunscreen and/or sunblock compounds, make-up agents, detergents, pharmaceutical drugs, emulsifiers, antiseptic agents, deodorant actives, dermatologically acceptable carriers, abrasives, absorbents, colorants, essential oils, skin sensates, cosmetic astringents, anti-acne agents, anti-caking agents, anti foaming agents, antioxidants, binders, biological additives, enzymes, enzymatic inhibitors, enzyme-inducing agents, coenzymes, plant extracts, plant derivatives, plant tissue extracts, plant seed extracts, plant oils, botanicals, botanical extracts, ceramides, peptides, buffering agents, bulking agents, chelating agents, chemical additives, colorants, cosmetic biocides, denaturants, drug astringents, external analgesics, film formers or materials, opacifying agents, pH adjusters, propellants, reducing agents, sequestrants, skin bleaching and lightening agents, skin tanning agents, skin-conditioning agents (e.g., humectants), skin soothing and/or healing agents and derivatives, thickeners, peeling agents, moisturizing agents, curative agents, lignans, preservatives, UV absorbers, a cytotoxic, an antineoplastic agent, a fat-soluble active, suspending agents, viscosity modifiers, diluents, pearlescent aids, foam boosters, or mixtures of any of the foregoing.

In certain embodiments, the skin care compositions and extracts discussed herein may be contacted with the skin of a patient—that is, a human or other mammal. Contact may be done simply by applying the skin care compositions or extracts manually, or with any personal care implement such as, for example, a brush, cotton ball, pad, paddle or swab. In certain embodiments, the compositions and extracts may be applied to the skin in the form of a mask, peel or wrap, and may be contacted with the skin for either a short period of time or a prolonged period of time to maximize effect.

In certain embodiments, the skin care compositions comprise an effective amount of such extracts, or an effective amount sufficient to prevent or reduce fibroblast collapse. As used herein, “reduce” means to decrease by any measurable amount. As used herein, “prevent” means to reduce by, in certain embodiments, at least about 5%, at least about 10%, at least about 20%, at least about 35%, at least about 50%, at least about 55%, at least about 60%, at least about 65%, at least about 70%, at least about 75%, at least about 80%, at least about 85% or at least about 90% of fibroblast collapse when compared to a control composition with no extract of Osmanthus, or with other extracts that are currently used in known skin compositions.

In certain embodiments, the compositions discussed herein are effective in reducing or preventing fibroblast collapse, in maintaining or increasing elasticity of the skin, in reducing the appearance of wrinkles or aging, or in prolonging the life of a fibroblast. As used herein, “prolong the life” means, in certain embodiments, lengthening the life of a fibroblast before its collapse by a time period of at least about 5%, at least about 10%, at least about 20%, at least about 35%, at least about 50%, at least about 55%, at least about 60%, at least about 65%, at least about 70%, at least about 75%, at least about 80%, at least about 85% or at least about 90% of the lifetime of a fibroblast that has not been contacted with an extract of Osmanthus.

As used herein, “reducing the appearance of aging” means reducing the visible appearance of aging on the skin of a patient, including but not limited to the appearance of certain aging indicators such as wrinkles, fine lines, laugh lines and furrows, spots (including age spots and freckles and moles), visible sun damage and the like, all indicators of the appearance of aging.

Herein, measurement of the amount of reduction in the appearance of aging can be made on a quantitative basis—for example:
measuring and comparing the number of aging indicators (e.g., measuring number of wrinkles or moles) before and after contacting the same area of skin with a skin care composition herein (for example, using laser scanning microscopy, ultrasound or merely the naked eye); or
measuring and comparing the number of aging indicators on a first sample of skin that has been contacted with the skin care composition herein with a second sample of skin that has had no such contact; or
measuring and comparing the intensity of the aging indicators (for example, measuring the depth of wrinkles or color intensity/contrast of age spots, moles or freckles before and after contacting the same area of skin with a skin care composition herein; or comparing the relative amounts of water retained in the same area of skin with a skin care composition herein), again, using, for example, laser scanning microscopy, ultrasound or merely the naked eye.

In various embodiments, the skin care compositions herein reduce the appearance of aging by at least about 5%, at least about 10%, at least about 15% or at least about 20% when compared with skin that has not been contacted with a skin composition herein.

In certain embodiments, the skin care compositions herein are effective at preventing glycation in amounts of at least about 5%, at least about 10%, at least about 20%, at least about 35%, at least about 50%, at least about 55%, at least about 60%, at least about 65%, at least about 70%, at least about 75%, at least about 80%, at least about 85% or at least about 90% when compared to a control composition with no extract of Osmanthus, or with other extracts that are currently used in known skin compositions. This can be measured, for example, by measuring the amount of glycation present in a first skin sample that has been contacted with a skin care composition as discussed herein, and comparing it to the amount of glycation present in a second skin sample that has not been contacted with a skin composition herein.

In certain embodiments, the skin care compositions herein maintain or increase elasticity of skin by at least about 5%, at least about 10%, at least about 20%, at least about 35%, at least about 50%, at least about 55%, at least about 60%, at least about 65%, at least about 70%, at least about 75%, at least about 80%, at least about 85% or at least about 90% when compared to a control composition with no extract of Osmanthus, or with other extracts that are currently used in known skin compositions.

In certain embodiments, the skin care compositions herein maintain or increase firmness of the skin of a patient by at least about 5%, at least about 10%, at least about 20%, at least about 35%, at least about 50%, at least about 55%, at least about 60%, at least about 65%, at least about 70%, at least about 75%, at least about 80%, at least about 85% or at least about 90% when compared to a control composition with no extract of Osmanthus, or with other extracts that are currently used in known skin compositions.

Both elasticity and firmness can be tested by, for example, cutometry, which is a mechanical method of applying a vacuum to the skin and measuring the pressure; or by use of a machine such as a ballistometer with a pendulum dropped from a fixed height onto the skin's surface with the firmness and/or elasticity recorded; or by any device that applies a torque or twisting motion to the skin, with the time for the skin to return to its normal state measured and recorded; or by measuring the resonance running time of an acoustical shockwave and determining collagen and elastin fibers as well as firmness and skin elasticity.

In various embodiments, the compositions discussed herein comprise an extract of a plant of genus Osmanthus as the primary active ingredient—that is, all remaining ingredients in such embodiments are either inert or have no appreciable effect on the skin. In certain embodiments, the majority (that is, greater than half) of any benefit to the skin can be attributable to the presence of the Osmanthus extract, in whole or in part.

In various embodiments, the skin care compositions discussed herein comprise about 0.001 to about 50% by weight of the Osmanthus extract. In various embodiments, the skin care compositions comprise about 0.001 to about 50%, about 0.001 to about 25%, about 0.001 to about 20%, about 0.001 to about 15, about 0.005 to about 5%, or about 0.01 to about 5%, or about 1%, about 2%, about 3% or about 4%, about 5% or about 10% by weight of Osmanthus extract.

In some examples discussed herein, comparison was made between the skin care compositions of the present technology and those containing pyridoxamine as a positive control. It was shown that a population of human dermal fibroblasts (HDFs) exposed to glycated proteins is generally expected to collapse. Specifically, glycated protein (such as bovine serum albumin, BSA), when added to normally cultured skin fibroblasts at a concentration in the range of 2 to 6 mg/mL, caused cell death within approximately 18 to 36 hours. Addition of pyridoxamine as a positive control for preventing glycation mitigated some of the collapse, but addition of the skin care compositions of the present technology protected the fibroblasts against the toxic effects of glycation in a linear dose response with nearly 100% protection when the composition comprises about 0.001 to about 15% by weight of Osmanthus.

In certain embodiments, the present compositions and methods are shown to be effective in preventing of at least about 5%, at least about 10%, at least about 20%, at least about 35%, at least about 50%, at least about 55%, at least about 60%, at least about 65%, at least about 70%, at least about 75%, at least about 80%, at least about 85% or at least about 90% of glycation when compared to a control composition with no extract of Osmanthus, or with other extracts that are currently used in known skin compositions. In certain embodiments, such compositions and methods are shown to be effective in preventing about 10 to about 95%, or about 20 to about 90%, or about 35 to about 85%, or about 50 to about 75% of glycation. In fact, in certain embodiments, compositions of the present technology when contacted with protein glycation mixtures (that also contained ribose, a glycating sugar) in amounts of about 0.001 to about 15%, were shown to prevent about 80% to about 95% of protein glycation; and the toxicity (lethality) of the protein when subsequently purified and added to skin fibroblast cultures was prevented by at least about 95%. Proteins containing advanced glycation end products are known to be toxic to fibroblasts.

The present technology is directed, in other embodiments, to compositions comprising an extract of Osmanthus in delivery systems such as, but not limited to, liposomes and proliposomes, micelles and promicelles, microcapsules, nanocapsules, millispheres, microspheres, sponges, nanospheres, lipospheres, microemulsions and nanoemulsions and the like. In certain embodiments, the skin care compositions can also be characterized as cosmetic or dermopharmaceutical compositions.

In certain embodiments, the skin care compositions discussed herein may contain a cosmetically or dermapharmaceutically effective amount of at least one compound or extract or oil with MMP and/or Elastase inhibition activity such as, e.g., Eugenia Caryophyllus (Clove) Flower Extract (Tellirictin®); Vaccinium Macrocarpon (Cranberry) Fruit Extract (Delphinitex®); Camellia Sinensis Leaf Extract (Scavenox™ GTE); Euterpe Oleracea Fruit Extract (and) Punica Granatum Extract (and) Glycerin (Ellagicin®); AFA Algae extract (DermalRx® KBGA).

In certain embodiments, the skin care compositions discussed herein may contain a cosmetically or dermapharmaceutically effective amount of at least one compound or extract or oil that have activity recovering and/or maintaining the barrier function of the skin such as, e.g., Yeast Extract (DermalRx® HydroSeal); Chondrus crispus Extract (and) Sodium Hyaluronate (MariMoist®).

In certain embodiments, the skin care compositions discussed herein may contain a cosmetically or dermapharmaceutically effective amount of at least one compound or extract or oil that have activity increasing collagen production by fibroblasts such as, e.g., Yeast Extract (DermalRx® HydroSeal); Yeast Extract (DermalRx® SRC).

In certain embodiments, the skin care compositions discussed herein may contain a cosmetically or dermapharmaceutically effective amount of at least one compound or extract or oil that have keratolytic and/or exfoliant and/or desquamating activity such as, e.g., Yeast Extract (DermalRx® SRC).

In certain embodiments, the skin care compositions discussed herein may further contain one or more of the following additional ingredients: sugar amines, glucosamine, D-glucosamine, N-acetyl glucosamine, N-acetyl-D-glucosamine, mannosamine, N-acetyl mannosamine, galactosamine, N-acetyl galactosamine, vitamin B3 and its derivatives, niacinamide, sodium dehydroacetate, dehydroacetic acid and its salts, phytosterols, salicylic acid compounds, hexamidines, dialkanoyl hydroxyproline compounds, soy extracts and derivatives, equol, isoflavones, flavonoids, phytantriol, farnesol, geraniol, peptides and their derivatives, di-, tri-, tetra-, penta-, and hexapeptides and their derivatives, lys-thr-thr-lys-ser, palmitoyl-lys-thr-thr-lys-ser, carnosine, N-acyl amino acid compounds, retinoids, retinyl propionate, retinol, retinyl palmitate, retinyl acetate, retinal, retinoic acid, water-soluble vitamins, ascorbates, vitamin C, ascorbic acid, ascorbyl glucoside, ascorbyl palmitate, magnesium ascorbyl phosphate, sodium ascorbyl phosphate, vitamins their salts and derivatives, provitamins and their salts and derivatives, ethyl panthenol, vitamin B, vitamin B derivatives, vitamin B1, vitamin B2, vitamin B6, vitamin B12, vitamin K, vitamin K derivatives, pantothenic acid and its derivatives, pantothenyl ethyl ether, panthenol and its derivatives, dexpanthenol, biotin, amino acids and their salts and derivatives, water soluble amino acids, asparagine, alanine, indole, glutamic acid, water insoluble vitamins, vitamin A, vitamin E, vitamin F, vitamin D, mono-, di-, and tri-terpenoids, beta-ionol, cedrol, and their derivatives, water insoluble amino acids, tyrosine, tryptamine, butylated hydroxytoluene, butylated hydroxyanisole, allantoin, tocopherol nicotinate, tocopherol, tocopherol esters, palmitoyl-gly-his-lys, phytosterol, hydroxy acids, glycolic acid, lactic acid, lactobionic acid, keto acids, pyruvic acid, phytic acid, lysophosphatidic acid, stilbenes, cinnamates, resveratrol, kinetin, zeatin, dimethylaminoethanol, natural peptides, soy peptides, salts of sugar acids, Mn gluconate, Zn gluconate, particulate materials, pigment materials, natural colors, piroctone olamine, 3,4,4′-trichlorocarbanilide, triclocarban, zinc pyrithione, hydroquinone, kojic acid, ascorbic acid, magnesium ascorbyl phosphate, ascorbyl glucoside, pyridoxine, aloe vera, terpene alcohols, allantoin, bisabolol, dipotassium glycyrrhizinate, glycerol acid, sorbitol acid, pentaerythritol acid, pyrrolidone acid and its salts, dihydroxyacetone, erythrulose, glyceraldehyde, tartaraldehyde, clove oil, menthol, camphor, eucalyptus oil, eugenol, menthyl lactate, witch hazel distillate, eicosene and vinyl pyrrolidone copolymers, iodopropyl butylcarbamate, a polysaccharide, an essential fatty acid, salicylate, glycyrrhetinic acid, carotenoides, ceramides and pseudo-ceramides, a lipid complex, oils in general of natural origin such shea butter, apricot oil, onagre oil, prunus oil, palm oil, monoi oil, HEPES; procysteine; O-octanoyl-6-D-maltose; the disodium salt of methylglycinediacetic acid, steroids such as diosgenin and derivatives of DHEA; DHEA or dehydroepiandrosterone and/or a precursor or chemical or biological derivative, N-ethyloxycarbonyl-4-para-aminophenol, bilberry extracts; phytohormones; extracts of the yeast Saccharomyces cerevisiae; extracts of algae; extracts of soyabean, lupin, maize and/or pea; alverine and its salts, including alverine citrate, extract of butcher's broom and of horse chestnut.

The present technology is directed, in further embodiments, to methods of reducing or preventing fibroblast collapse by applying compositions discussed herein to the skin of a patient (including, in certain embodiments, the hair of a patient). As used herein, “patient” may refer to any human or animal having a skin. In various embodiments, the patient may be a human or a mammal.

The present technology is directed, in further embodiments, to a method of improving a composition's efficacy in reducing or preventing fibroblast collapse, the composition comprising an extract of a plant of genus Osmanthus, wherein the method comprises optimizing the concentration and amount of the Osmanthus extract therein.

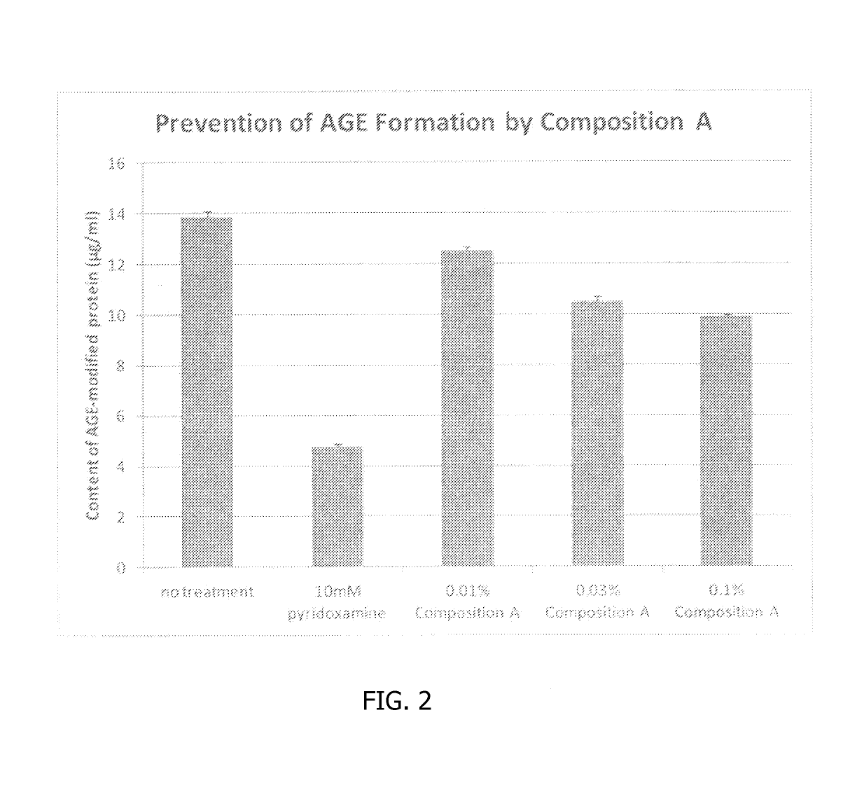

Certain embodiments of the present technology are shown in the attached Figures. For example, FIG. 2 is a bar graph that shows that by preventing glycation of the target protein (BSA=bovine serum albumin), the skin care compositions of the present technology prevent the target protein from becoming toxic to skin fibroblasts. It is known that protein that is modified with advanced glycation end products (AGEs), the end result of glycation, is toxic to fibroblasts.

### Example 1

An extract was prepared as follows:

1. Parts of the dried Osmanthus plant in an amount of about 1 to about 5 grams (in various embodiments, about 1, about 2, about 3, about 4 or about 5 grams) is combined with about 95 to about 99 grams of deionized water (amount dependent on amount of dried Osmanthus plant, such that the total=100 grams).

2. This was heated for about 2 to about 6 hours at about 40 to about 80° C. The material was then cooled and filtered to remove suspended particulates.

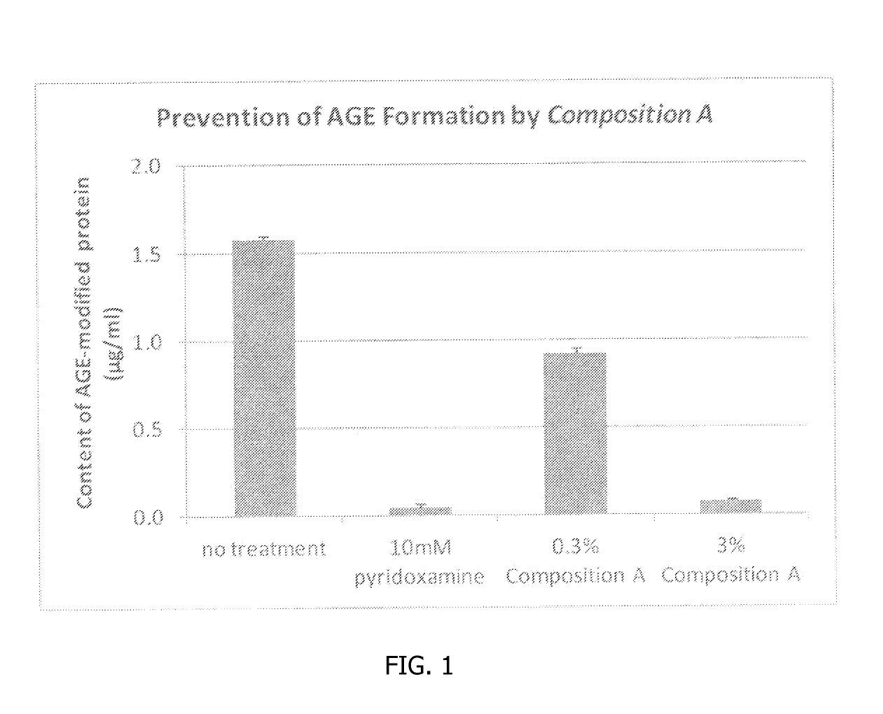

3. The remaining solution (referred to herein as “Composition A”) was applied to a target protein in the presence of a glycating sugar and compared with the following controls, as shown in FIG. 1: no treatment (i.e., target protein plus sugar, without any other agent present), 10 mM pyridoxamine (i.e., target protein plus sugar plus 10 mM pyridoxamine). As can be seen in FIG. 1, when the AGE contents of the variously-treated protein mixtures were determined by enzyme-linked immunoassay, the composition comprising the Osmanthus extract showed marked dose-dependent reduction in the content of AGE-modified protein.

In modifications of this example, the amount of Osmanthus could be varied as discussed above; for example, in amounts of anywhere from 1 to 99 parts, with the amount of water or other solvent varying to bring the entire composition to 100 parts.

### Example 2

An extract was prepared as follows:

1. Parts of the dried Osmanthus plant in an amount of about 1 to about 5 grams is combined with about 95 to about 99 grams of deionized water (total=100 grams).

2. This was heated for about 2 to about 6 hours at about 40 to about 80° C. The material was then cooled and filtered to remove suspended particulates.

3. The remaining solution (referred to herein as “Composition A”) was applied to a target protein in the presence of 10 millimolar glyoxal, a reactive carbonyl compound, and compared with the following controls, as shown in FIG. 2: no treatment (i.e., target protein plus glyoxal, without any other agent present), 10 mM pyridoxamine (i.e., target protein plus glyoxal plus 10 mM pyridoxamine). As can be seen in both FIG. 1 and FIG. 2, when the AGE contents of the variously-treated protein mixtures were determined by enzyme-linked immunoassay, the composition comprising the Osmanthus extract showed statistically significant dose-dependent reduction in the content of AGE-modified protein.

### Example 3

A series of tests was conducted to evaluate the skin care compositions of the present technology, to see whether they could prevent the target protein of BSA from being toxic to skin fibroblasts. This was done by first reacting BSA with the sugar (ribose) in a buffered saline solution. Some of the reaction mixtures also contained pyridoxamine (a drug used here as the positive control known to prevent glycation) or the skin care compositions of the present technology.

For this test, incubation of the sterile reaction mixtures was for 6.5 days at a temperature of about 45° C. After that, the reaction mixtures were extensively dialyzed against deionized water, removing all of the small molecules (ribose, salts, etc.) from the mixtures, leaving just the BSA. The resulting BSA solutions were then lyophilized (freeze-dried) to remove water, and the residues of BSA were resolubilized in a buffered saline solution. After measuring the protein concentration of each of these solutions, they were each added to several replicate wells of a multiwell cell culture plate containing fibroblasts (same number of cells in all wells), at a final BSA concentration of 5.5 milligrams per milliliter.

After exposure of the cells to these solutions at 37° C. for 24 hours, the viability of the cells was determined by adding a dye (resazurin) to the wells and incubating at 37° C. for another hour. Live cells metabolize the dye and it changes color, dead cells do not change it. The color change in the wells was then measured as optical density on a plate-reading spectrophotometer. The optical density (cell viability) in each case is normalized by reference to that for the “no protein (control)” condition which is set to 100%. The specific meaning of the graph labels are as follows:

No Protein (Control):

These are cells to which the buffered saline was added without any BSA in it. These cells show full (100%) viability.

Protein Alone (No Sugar):

These are cells to which unglycated BSA in buffered saline was added. This is BSA that was never incubated with ribose.

Protein+Sugar:

These are cells incubated with buffered saline that contained BSA that had been reacted with 100 millimolar ribose. This is highly-glycated BSA. The percent viability is low, meaning that most of the cells were killed by this BSA sample.

100 mM Ribose+1% Composition A:

These are cells that were incubated with buffered saline that contained BSA that had been reacted with 100 millimolar ribose in the presence of 1% of Composition A. The Osmanthus extract prevents glycation so that this BSA sample has very little toxicity. In fact, the viability here is about 95% and is not statistically different than the no BSA control. Thus, the protection from fibroblast collapse is superior.

### Example 4

Cream formulations were made in accordance with certain embodiments of the technology. “Composition A” refers to a composition in accordance with the present technology that contains the extract of Osmanthus. The following ranges of ingredients were included:

Emulsifiers:

about 0.1 to about 99%, about 0.1 to about 20%, about 1 to about 20, about 1.5 to about 10% or about 2 to about 6%.

Emollients:

about 0.1 to about 99%, about 0.1 to about 60%, about 1 to about 60%, about 5 to about 50% or about 10 to about 35%.

Thickeners:

about 0.01 to about 10%, about 0.01 to about 8%, 0.01 to about 5%, about 0.05 to about 5% about 0.1 to about 5%, about 0.1 to about 2% or about 0.1 to about 1%.

Water:

about 0.1 to about 99%, about 10 to about 95%, about 15 to about 90%, about 20 to about 80%, about 30 to about 90%, about 35 to about 90%, about 40 to about 90% or about 45 to about 80%.

Preservatives:

about 0.01 to about 5%, about 0.01 to about 2%, about 0.1 to about 1.5%, about 0.2 to about 1% or about 0.25 to about 0.75%

Composition A (Containing Osmanthus Extract):

about 0.01 to about 10%, about 0.1 to about 8%, about 0.1 to about 5% or about 1 to about 3%.

### Example 5

A cream composition was prepared in accordance with the following formula and method:

| Phase | Ingredient | % | INCI Names |
|---|---|---|---|
| A | Deionized Water | QS | Water |
| | Disodium EDTA | about 0.01 to about 1 | Disodium EDTA |
| B | Carbopol ® 980 Polymer | about 0.1 to about 1 | Carbomer (Lubrizol) |
| | Carbopol ® Ultrez-21 | about 0.1 to about 1 | Acrylates/C10-30 Alkyl |
| | | | Acrylate Crosspolymer |
| | | | (Lubrizol) |
| C | Butylene Glycol | about 1 to about 5 | Butylene Glycol |
| | Keltrol ® CG-RD | about 0.1 to about 1 | Xanthan Gum (CP Kelco) |
| D | Glycerin | about 1 to about 10 | Glycerin |
| | BPA-500X | about 0.5 to about 5 | Methyl Methacrylate |
| | | | Crosspolymer (Kobo |
| | | | Products) |
| E | Montanov ™ 68 | about 1 to about 5 | Cetearyl Alcohol, Cetearyl |
| | | | Glucoside (Seppic) |
| | Jeechem GMS-165 | about 1 to about 5 | Glyceryl Stearate, PEG-100 |
| | | | Stearate (Jeen Int'l Corp.) |
| | Schercemol ™ 1688 Ester | about 1 to about 10 | Cetearyl Ethylhexanoate |
| | | | (Lubrizol) |
| | Jeechem CTG | about 1 to about 10 | Caprylic/Capric Triglyceride |
| | | | (Jeen Int'l Corp.) |
| F | Dow Corning ® 1413 | about 1 to about 10 | Dimethicone (Dow Corning) |
| | Fluid | | |
| | Composition A | about 0.001 to about 20 | Water, Osmanthus Fragrans |
| | (containing Osmanthus | | Flower Extract, Propanediol, |
| | extract) | | Glycerin |
| | DermalRx ® HydroSeal | about 1 to about 10 | Water, Yeast Extract, Soy |
| | | | Amino Acids (Biocogent) |
| | HEDI ™ Shea Butter | about 1 to about 10 | Water, Dimethicone, |
| | E/DU | | Butyrospermum Parkii (Shea |
| | | | Butter) Extract, Lecithin, |
| | | | Urea, Tocopherol Acetate |
| | | | (Biocogent) |
| | Phenoxyethanol | about 0.1 to about 1 | Phenoxyethanol |
| | Symdiol ™ 68 | about 0.5 to about 5 | 1,2-Hexanediol, Caprylyl |
| | | | Glycol (Symrise) |
| | Fragrance | about 0.01 to about 1 | Fragrance |
| G | Deionized Water | about 0.5 to about 5 | Water |
| | Potassium Sorbate | about 0.1 to about 1 | Potassium Sorbate |
| H | NaOH 30% Aqueous | QS | Water, Sodium Hydroxide |
| | Solution | | |

Manufacturing Procedure:
1. Phases A, B, C, D and E were propeller mixed, heated to a range of about 70 to about 80° C., and processed with a rotor-stator emulsifier.
2. When the mixed batch had been thoroughly emulsified, the mixture was cooled to a range of about 40 to about 50° C.
3. Phase F, G and H ingredients were then added, and the resultant mixture was thoroughly mixed.

### Example 6

A serum composition was prepared in accordance with the following formula and method:

| Phase | Ingredient | % | INCI Names |
|---|---|---|---|
| A | Deionized Water | about 50 to about 90 | Water |
| B | Sepinov EMT-10 | about 0.5 to about 5 | Hydroxyethyl acrylate/sodium |
| | | | acryloyldimethyl taurate copolymer |
| C | Butylene Glycol | about 1 to about 10 | Butylene Glycol |
| | Glycerin | about 1 to about 10 | Glycerin |
| | Keltrol CG RD | about 0.1 to about 5 | Xanthan Gum |
| D | Xiameter ® PMX-200 Silicone | about 1 to about 10 | Dimethicone |
| | Fluid, 20CS | | |
| | Diocide | about 0.5 to about 5 | Caprylyl Glycol, Phenoxyethanol, |
| | | | Hexylene Glycol |
| | Composition A (containing | about 0.001 to about 20 | Water, Osmanthus Fragrans Flower |
| | Osmanthus extract) | | Extract, Propanediol, Glycerin |

Manufacturing Procedure:
1. Phases A, B, C were propeller mixed until uniform.
2. Phase D was then added. Mixture was processed with rotor-stator emulsifier and homogenized.

### Example 7

A further series of tests was conducted to evaluate the skin care compositions of the present technology, to see whether they could prevent the target protein of BSA from being toxic to skin fibroblasts. This was done by first reacting BSA with the glycating sugar (ribose) in a buffered saline solution. Some of the reaction mixtures also contained pyridoxamine (a drug used here as the positive control known to prevent glycation) or the skin care compositions of the present technology.

For this test, incubation of the sterile reaction mixtures was for two days at a temperature of about 45° C. After that, the reaction mixtures were extensively dialyzed against deionized water, removing all of the small molecules (ribose, salts, etc.) from the mixtures, leaving just the BSA. The resulting BSA solutions were then lyophilized (freeze-dried) to remove water, and the residues of BSA were resolubilized in a buffered saline solution. After measuring the protein concentration of each of these solutions, they were each added to a cell culture dish containing confluent fibroblasts to achieve a final BSA concentration of 2 milligrams per milliliter.

After exposure of the cells to these solutions at 37° C. for 16 hours, the viability of the cells was determined by adding a dye (neutral red) to the plates and incubating at 37° C. for another hour. Live cells take up and concentrate the dye inside their lysosomes thereby appearing red, whereas dead cells do not sequester the dye. The cells were then photographed under a microscope using brightfield optics. The specific meaning of the photograph labels and interpretation of the results are as follows:

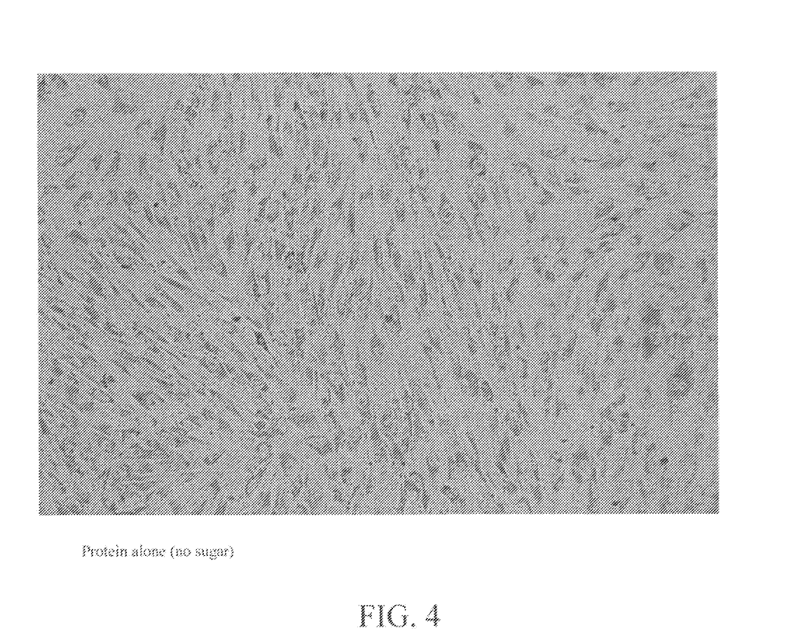

FIG. 4 Protein Alone (No Sugar):

These cells were exposed to BSA which had not been reacted with ribose. The cells are stained because they have accumulated the neutral red dye, and they appear well-attached to, and spread out on the culture dish surface, all indications of healthy cells.

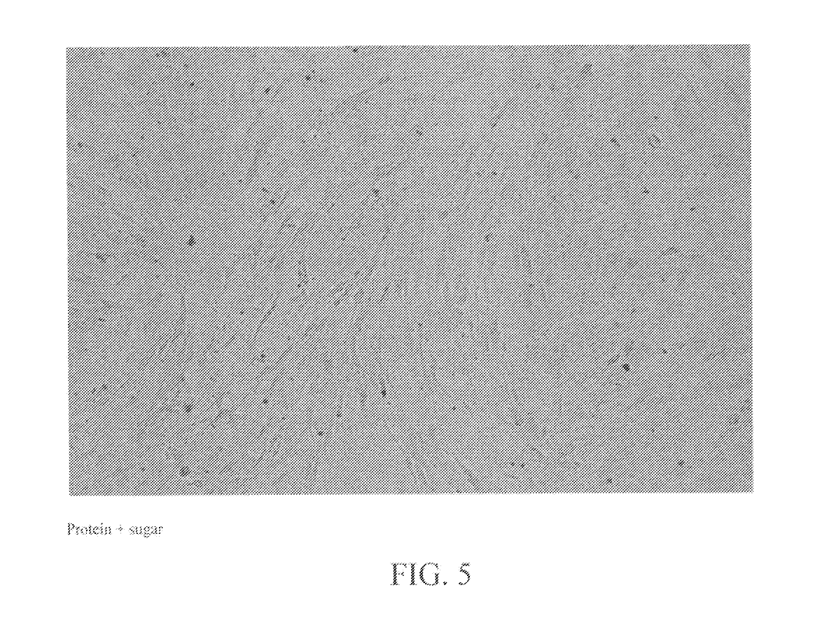

FIG. 5 Protein+Sugar:

These cells were exposed to BSA which had been reacted with 100 millimolar ribose. These cells are not stained because they are metabolically inactive (dead) and do not accumulate the dye. They have also lost most of their strong attachment to the dish surface.

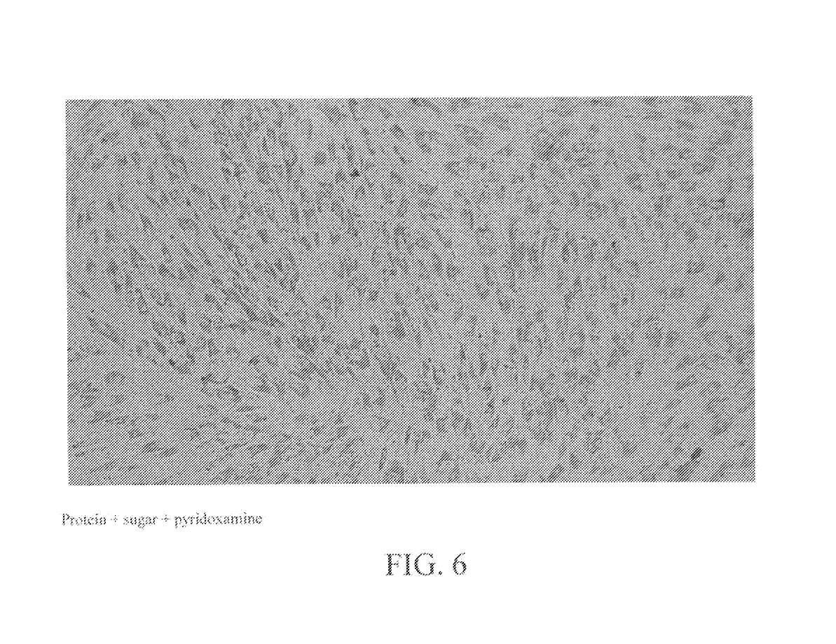

FIG. 6 Protein+Sugar+Pyridoxamine:

These cells were exposed to BSA which had been reacted with 100 millimolar ribose in the presence of 10 millimolar pyridoxamine, an inhibitor of glycation. These cells have remained healthy and have accumulated the dye and have remained attached to the culture dish surface.

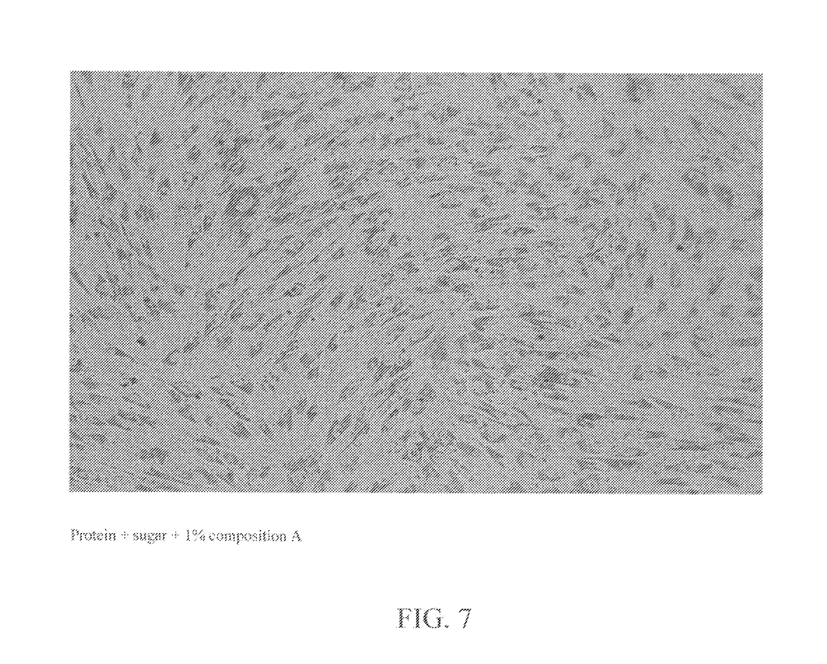

FIG. 7 Protein+Sugar+1% Composition A:

These cells were exposed to BSA that had been reacted with 100 millimolar ribose in the presence of 1% of Composition A. Composition A prevents glycation so that this BSA sample has very little toxicity. As such, the cells have accumulated dye and have remained attached to the dish surface, signs of a healthy culture. Thus, the prevention by Composition A of fibroblast collapse is highly effective.

Although the present technology has been described in relation to particular embodiments thereof, these embodiments and examples are merely exemplary and not intended to be limiting. Many other variations and modifications and other uses will become apparent to those skilled in the art. The present technology should, therefore, not be limited by the specific disclosure herein, and may be embodied in other forms not explicitly described here, without departing from the spirit thereof.

What is claimed is:

1. A skin care composition comprising an extract of a plant of genus Osmanthus.

2. The skin care composition of claim 1, wherein the plant is chosen from Osmanthus americanus, Osmanthus amatus, Osmanthus asiaticus (sweet olive), Osmanthus auratiacus, Osmanthus decorus, Osmanthus delavayi, Osmanthus fragrans, Osmanthus heterophyllus, Osmanthus semulatus, Osmanthus suavis, Osmanthus yunnanensis, Osmanthus×burkwoodii (O. delavayi×O. decorus) and Osmanthus×fortunei (O. fragrans×O. heterophyllus).

3. The skin care composition of claim 1, wherein the extract is in the form of a powder or pulverized material, granule, tablet, a cream, grounds, an emulsion, a suspension or paste.

4. The skin care composition of claim 1, wherein the skin care composition is in the form of a lotion, a cream, a paste, a suspension, a gel, a liquid, an aerosol, a powder, a foam, a serum, an ointment or mixture of any of the foregoing.

5. The skin care composition of claim 1, further comprising at least one additional skin care ingredient chosen from an emollient, a surfactant, a solvent, a vitamin, a lipid, a sunscreen agent, a polymer, a moisturizer, a colorant, a maskant, a fragrance, a preservative or an exfoliating agent.

6. The skin care composition of claim 1, wherein the extract is present in an amount of about 0.001 to about 15% by weight of the skin care composition.

7. A skin care composition comprising:
(a) about 0.001 to about 50% of an extract of a plant of genus Osmanthus;
(b) about 0.1 to about 20% of an emulsifier;
(c) about 0.1 to about 60% of an emollient;
(d) about 0.01 to about 10% of a thickener; and
(e) about 0.1 to about 99% water.

8. The skin care composition of claim 7, further comprising at least one additional skin care ingredient.

9. The skin care composition of claim 8, wherein the skin care ingredient is chosen from an emollient, a surfactant, a solvent, a vitamin, a lipid, a sunscreen agent, a polymer, a moisturizer, a colorant, a maskant, a fragrance, a preservative or an exfoliating agent.

10. A method of formulating a skin care composition, the method comprising the steps of:
(a) preparing an extract of a plant of genus Osmanthus by preparing a combination of about 2 to about 10 parts by weight of any portion of the plant with about 90 to about 98 parts by weight of solvent; and
(b) combining the extract with a skin care ingredient to produce the skin care composition.

11. The method of claim 10, wherein between steps (a) and (b) the extract is turned into a powder or pulverized material, granule, tablet, a cream, grounds, an emulsion, a suspension or paste.

12. A method of applying a skin care composition according to claim 1 to the skin of a patient.

13. A method of reducing or preventing fibroblast collapse in the skin of a patient, the method comprising the step of contacting the skin care composition of claim 1 with the skin of a patient.

14. The method of claim 13, wherein the fibroblast collapse is reduced or prevented by an amount of at least about 5% when compared with the amount of fibroblast collapse on the skin of a patient that has not been contacted with the skin care composition.

15. A method of reducing the appearance of aging on the skin of a patient, the method comprising the step of contacting the skin care composition of claim 1 with the skin of a patient;
wherein the appearance of aging on the skin of the patient is reduced by at least about 5% when compared with the appearance of aging on the skin on the patient that has not been contacted with the skin care composition.

16. A method of preventing glycation of a protein in the skin of a patient, the method comprising the step of contacting the skin care composition of claim 1 with the skin of a patient;
wherein glycation of the protein is prevented by at least about 5% when compared with the glycation of a the protein in the skin of a patient that has not been contacted with the skin care composition.

17. A method of maintaining or increasing elasticity in the skin of a patient, the method comprising the step of contacting the skin care composition of claim 1 with the patient; wherein the elasticity of the skin of the patient is maintained or increased by at least about 5% when compared with the elasticity of the skin of a patient that has not been contacted with the skin care composition.

18. A method of maintaining or increasing firmness of the skin of a patient, the method comprising the step of contacting the skin care composition of claim 1 with the patient; wherein the firmness of the skin of the patient is maintained or increased by at least about 5% when compared with the firmness of the skin of a patient that has not been contacted with the skin care composition.
